(19) United States
(12) Patent Application Publication
(10) Pub. No.: US 2007/0142809 A1
(43) Pub. Date: Jun. 21, 2007

(54) Baby product

(76) Inventor: Elizabeth Pimm, Cambridge (GB)

Correspondence Address: John B Hardaway III;Nexsen Pruet Adams Kleemeier, P O Box 10107, Greenville, SC, 29603, US

(21) Appl. No.: 10/587,787

(22) PCT Filed: Jan. 28, 2005
(86) PCT No.: PCT/GB05/00300

§ 371 Date: Jul. 28, 2006

(30) Foreign Application Priority Data
Jan. 30, 2004 (GB) 0402126.7

(51) Int. Cl. A61F 13/15

(52) U.S. Cl. 604/385.06; 604/385.02; 206/440

(57) ABSTRACT

The invention described is a single-use kit (1) for hygienically changing and disposing of a baby's nappy. The kit in one aspect comprising a foldable and/or rollable mat (3) and a first mat extension (4) having a pocket (6) containing a nappy-changing material. The kit is openable by unfolding and/or unrolling the mat and first mat extension and closable by folding and/or rolling the mat and first mat extension. When open, the kit when open provides (i) a nappy-changing surface on the mat and (ii) single-handed access to the nappy-changing material. When closed, the kit is suitable for enclosing a soiled nappy and/or used nappy-changing material, allowing for convenient and hygienic disposal.

The present invention relates to a product for use with babies, in particular a kit for changing a baby's nappy.

Travelling with or looking after a baby away from home generally requires several baby accessories which are often carried in a bag. Typical accessories include some sort of changing mat, one or more unused nappies, a container of baby-wipes, one or more containers of nappy cream and/or baby powder, a sealable container for soiled nappies, bottles of water, juice or milk, rusks or baby snacks, and several favorite rattles or toys. It may take some time to locate one or more of the required accessories, and together they can be bulky and inconvenient to carry around.

For example, walks or shopping with a baby can become an epic journey for a baby carer, requiring a bulky accessories bag to squeezed into (and possibly overbalance) a pushchair, take up space under a car seat or other seat, and/or be carried with some difficulty in addition to the baby.

Several public establishments do provide facilities for baby changing, for example a changing mat and a nappy disposal bin, and this may partially alleviate the requirement for certain baby accessories at least in an urban environment. However, the use of such public facilities may not be convenient or hygiene standards may be inadequate.

The prior art provides several examples of nappy changing aids. For instance, U.S. Pat. No. 4,154,323 discloses a carrying bag for holding diapers and other baby supplies and incorporating a strip of material permanently or detachably secured to an inner portion of the bag. The strip of material can be extended from the top of the bag and used as a surface for changing a baby's nappy. GB2378380 teaches the use of a mat comprising pockets for nappies and the like and which can be rolled into a tubular configuration for storage or transport. In U.S. Pat. No. 4,886,150, a baby accessory carrier which doubles as a bag for accessories to feed and change a baby is described. The carrier comprises an elongated central changing area bordered by a pair of pocket-containing ends, and can be folded double to form an article more suitable for carrying. The device is intended to carry all of the accessories commonly associated with baby changing as well as bottles and other baby feeding equipment. U.S. Pat. No. 5,692,257 discloses a combination baby blanket, bunting and bag. The product is made of a soft cloth panel with pocket means for baby accessories. The cloth panel opens to form a large, soft baby blanket and is foldable to form a baby bunting or a bag.

Disposable nappies as well as disposable packages for receiving and retaining nappy-changing toiletries are also known. For example, U.S. Pat. No. 4,702,378 discloses a disposable kit having a body with several pockets for retaining sundry products for the care of a baby (such as toiletries and a nappy). The body can be folded so that the toiletries and nappy are enveloped within the interior of the kit and protected from degradation. The kit may be adapted to receive men's or women's toiletries, travel accessories or first aid supplies. U.S. Pat. No. 5,443,161 describes a disposable pack having supplies for two complete nappy changes. The pack is enclosed by material including a tear line to permit access to the supplies. GB2238286 discloses a disposable pack of items comprising an outer flat envelope perforated along one edge and containing a folded changing mat, nappy-changing materials and a disposal bag. After use, any soiled items can be returned to the disposal bag and discarded. GB2271720 discloses a disposable sanitary article such as a nappy with an attached releasable bag for enclosing a cleaning item.

While several nappy-changing articles have been developed, there remains a need for improvement. For example, in some cases prior art articles may not be particularly easy to handle while attending to a baby. Other articles may provide certain baby changing materials but not provide a suitable surface on which to change a nappy. Yet other articles may become unhygienic after repeated use. The present invention aims to provide an alternative and improved means for changing a baby's nappy.

According to the present invention there is provided a single-use kit for hygienically changing and disposing of a baby's nappy, comprising a foldable and/or rollable mat and a first mat extension having a pocket containing a nappy-changing material, in which the kit is openable by unfolding and/or unrolling the mat and first mat extension and closable by folding and/or rolling the mat and first mat extension, the kit when open providing (i) a nappy-changing surface on (or formed by) the mat and (ii) single-handed access to the nappy-changing material, and in which the kit when closed is suitable for enclosing a soiled nappy and/or used nappy-changing material.

The present invention thus provides a single-use nappy-changing kit (or packet) for short-term, emergency or regular use. The all-in-one kit can be used to easily and hygienically change a soiled nappy of a baby. The kit provides its own nappy-changing surface, allowing the kit to be used on substrates which may otherwise be unsuitable for changing a baby, for example a floor, a carpet, a table, grass or a seat of a car, aeroplane, train or other form of private or public transport. The kit may be used on unclean substrates such as a public baby changing area with inadequate hygiene standards.

The kit also provides a convenient vehicle for discarding the used nappy-changing surface, the soiled nappy and used nappy-changing material. The compactness of the mat and mat extension, and the ability to access nappy-changing material single-handedly makes the kit less fussy and easier to use than known nappy-changing articles.

The kit is “single-use” in that it is designed to be disposed after use. The perceived and actual value of the product will thus not be so high as to inhibit the disposal of the used product. The single-use kit is a low cost-per-unit solution for hygienically changing and disposing of a baby's nappy.

The kit may further comprise an unused nappy. The unused nappy and/or further nappy changing material may be positioned within the kit to be accessible when the kit is open. The nappy and/or further nappy changing material are in one embodiment loose within the kit so that they can be removed when the kit is open and before the baby is placed on the mat. The nappy is preferably a standard size, which can vary according to the age of the baby. The kit itself may be provided in different sizes for different baby sizes or ages.

In one embodiment, the pocket is frangibly sealed to facilitate single-handed opening. The pocket may comprise a tear strip (for example, located along one edge of the pocket) and/or a tab (for example, located at a corner of the pocket) to facilitate single-handed opening. Opening of the pocket may be assisted by the weight of the baby on the mat when the kit is being used. The mat extension may comprise two or more pockets containing different nappy-changing material.

The pocket may comprise two sides, a first side and a second side preferably formed by the mat extension. At least a portion of the first side may be attached to the second side using an easy-release adhesive, sealant, snap fastener, zipper, tie-means and/or hook-and-loop fastening means such as Velcro®. Nappy-changing material contained within the pocket may thus be easily accessible by peeling the first side away from the second side, thereby allowing access to the content of the pocket. A tab integral with the first side may be provided for this purpose. The weight of baby on the mat may provide leverage and/or stability to facilitate access to the nappy-changing material.

In a preferred embodiment, the pocket in a closed position is moisture-proof. This will be particularly useful for wet or liquid nappy-changing material.

The mat is preferably substantially rectangular when the kit is open, providing a convenient nappy-changing surface for the baby.

In one embodiment, the first mat extension (or flap) extends from a side of the mat. The kit preferably comprises a second mat extension (or flap) substantially opposite the first mat extension. The second mat extension may comprise the or further pockets. With two mat extensions, the kit when open may be of cruciform shape or be T-shaped. Furthermore, the kit may be closed by folding and/or rolling the mat and mat extensions, and opened by unfolding and/or unrolling the mat and mat extensions, the second mat extension forming a surface for receiving a soiled nappy when the kit is open.

The nappy-changing material and further nappy-changing material may include one or more of the following: one or more sheets of liquid-absorbing material (such as “dry wipes”), one or more sheets of material impregnated with a cleansing liquid, one or more sheets of material impregnated with an antiseptic and/or antiseptic material, a protective cream or lotion for a baby, hand cream, a bag (for example, a sealable bag such as a sealable plastic bag), and a baby toy (preferably a flat baby toy such as a teething ring). Nappy-changing material and particularly further nappy-changing material, for example liquid-based or liquid-containing further nappy-changing material, may optionally themselves be sealed in an openable container such as a plastic bag.

In a preferred embodiment, the sheets of material impregnated with a cleansing liquid or an antiseptic and/or antiseptic material, the protective cream or lotion for a baby, or a hand cream are contained with the pocket or pockets.

The sheets of material impregnated with a cleansing liquid or an antiseptic and/or antiseptic material may alternatively be positioned within the kit to be immediately accessible when the kit is open or partially open. The sheets of liquid-absorbing material, for example “dry wipes”, the bag or a baby toy, and/or a nappy, are preferably positioned within the kit to be immediately accessible when the kit is open or partially open. Upon opening the kit, such items may conveniently be placed on the first or second mat extension until needed.

The mat may comprise a padded section onto which the baby's head can be placed when the kit is open. This is particularly useful where a nappy change is required but no soft surfaces are available on which to place the kit.

The kit may comprise a fastener (for example, a releasable fastener such as a tie means and/or a hook-and-loop fastening means such as Velcro® and/or reusable adhesive tabs) attached to the mat and/or either mat extension, in which the fastener allows the kit to be held closed.

The kit may comprise a carry means (for example, a handle). This will make the kit more accessible and easier to dispose of.

The mat and/or mat extensions may be formed from a plastics material. Alternatively, the mat and/or mat extensions may be formed from paper with a plastics coating. The kit is preferably substantially biodegradable.

In a preferred embodiment, the kit, for example when closed, may be suitable for vending from a vending machine. The kit when closed may be of a compact and robust design to allow vending from a vending machine, for example from a vending machine at locations such as airports, train stations, bus stations, public toilets, or retail outlets. The kit when closed may have a waterproof outer surface, particularly when provided in a public vending machine.

In one aspect of the invention, the kit and/or its components are sterilisable or sterilised (for example, by irradiation and/or autoclaving). The kit may be sealed in a container, and/or the kit itself may be sealed when closed, so that it remains sterile until the container or kit are opened for use.

In one embodiment, the mat comprises a releasable layer (for example, a pad or sheet of impermeable material) covering at least part of the nappy-changing surface. This would allow the releasable layer to be used as a surface for removing a soiled nappy, following which the releasable layer could be removed to leave a clean surface on the mat for putting on a clean nappy or clean outer garments.

The invention also provides a method of producing the kit as described herein, and an apparatus for producing the kit. The kit may be produced using materials as described above. In one embodiment, the mat and mat extensions are manufactured (preferably in bulk) as one unit, with further components such as the nappy-changing material and further nappy-changing material then added to the pockets before the pockets are optionally sealed or placed within the mat prior to folding the mat. Components of the kit may be ultrasonically sealed.

Embodiments of the invention will now be described, by way of example only, with reference to the accompanying drawings, in which:

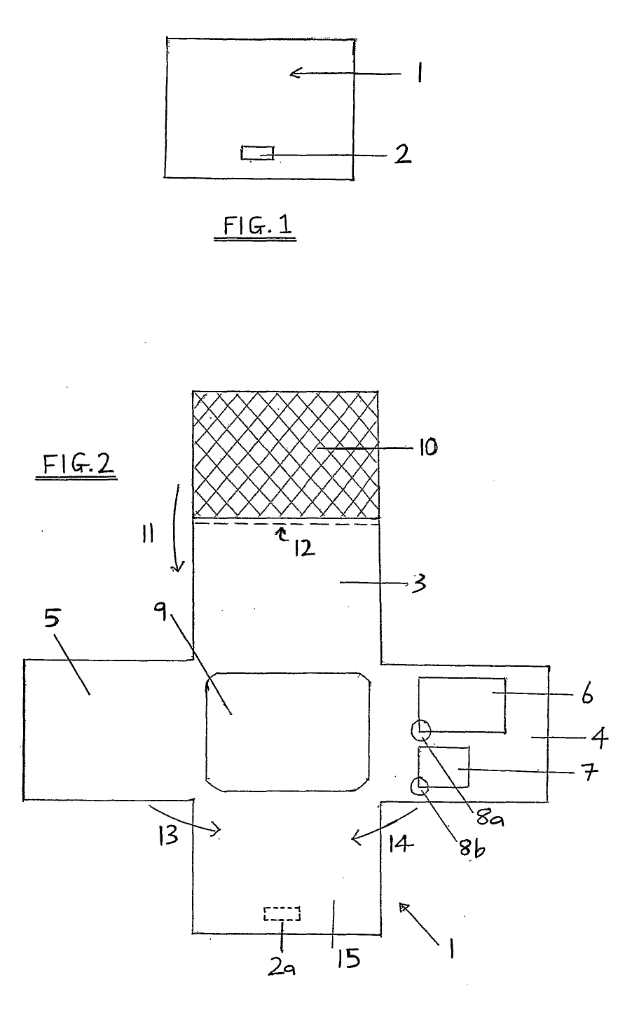

FIG. 1 is a plan view of a single-use nappy kit according to a first embodiment of the invention in a closed position;

FIG. 2 shows the kit of FIG. 1 in an open position;

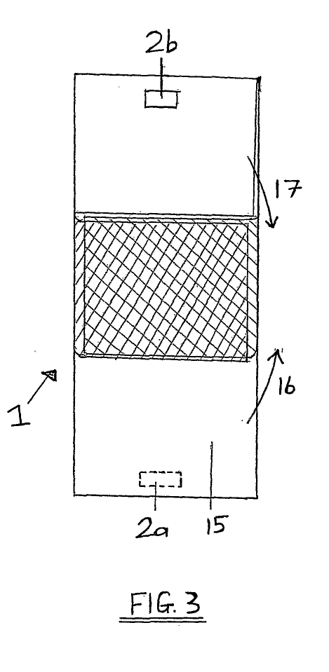

FIG. 3 shows the kit of FIGS. 1 and 2 in a partially closed position; and

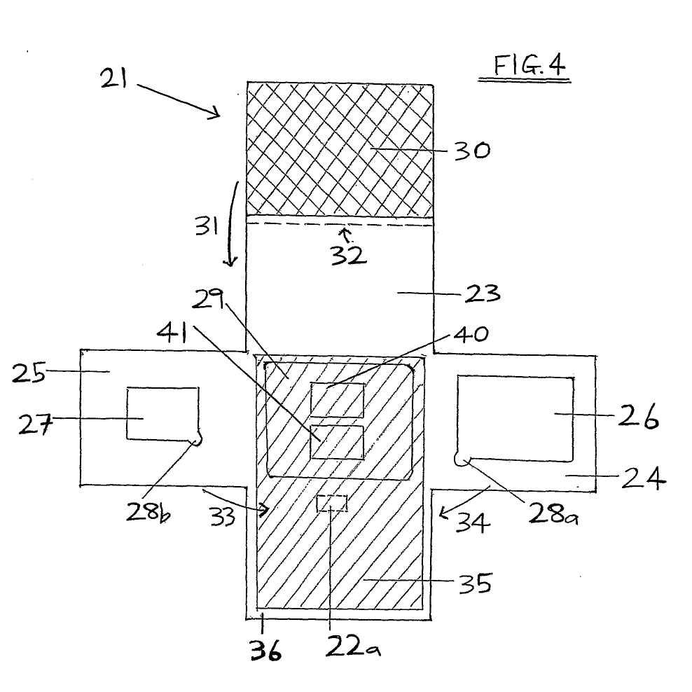

FIG. 4 shows a single-use nappy kit according to a second embodiment of the invention in an open position.

A nappy-changing kit 1 is shown in a closed position in FIG. 1. In the closed position, kit 1 is rectangular in plan view. The kit 1 is kept closed by a hook-and-loop fastener 2.

In the open position shown in FIG. 2, kit 1 is (small) T-shaped (inverted) with an elongated mat 3 having extending therefrom side flaps (or mat extensions) 4, 5. Side flap 4 incorporates a first envelope or pocket 6 with one or more sheets of a material moistened with cleansing liquid (for example, “baby wipes”) and a second envelope or pocket 7 with one or more antiseptic wipes for a carer to use while or after changing a nappy. Each pocket 6, 7 can be opened with one hand using a tear-tab 8a, 8b located at one corner of each pocket 6, 7. An unused nappy 9 (shown in outline) fits loosely onto a central position of the mat 3 and is available when the kit 1 is open. At an upper end of the mat 3 is located a padded head rest 10 onto which a baby's head can be rested.

In use, a basic nappy-changing kit 1 would first be opened following a reverse procedure to the closing sequence described below. When open, the kit 1 provides a clean, hygienic, elongated working surface on mat 3. The unused nappy 9 is set aside from the mat 3 and the baby reclined onto mat 3 with his or her head resting on head rest 10. The two flaps 4, 5 provide a wider working surface area. The pockets 6, 7 on one flap 4 are used to store materials required to change the baby, and these pockets 6, 7 are easily opened with one hand using the tear-tabs 8a, 8b so that the carer has a free hand for attending to the baby or soiled nappy. A soiled nappy once removed from the baby can be conveniently placed to one side on the other flap 5. The baby can then be cleaned using baby wipes provided in pocket 6, and the used wipes placed to one side on the other flap 5. The carer can wipe his or her hands using the antiseptic wipe provided in pocket 7, and used antiseptic wipes can also be placed on the other flap 5. The unused nappy can then be fitted onto the baby. The carer could again wipe his or her hands using a further antiseptic wipe provided in pocket 7, which can be placed on the other flap 5.

When finished with a nappy change, the kit 1 may be closed by folding the mat 3 and flaps 4, 5 as shown in FIG. 1. Before the folding sequence is commenced, the carer places the soiled nappy, used baby wipes and used antiseptic wipe into the central portion of the mat 3 between the flaps 4, 5. The upper end of the mat 3 extending beyond the flaps 4, 5 is folded inwardly and roughly in half (as indicated by arrow 11 in FIG. 1). Fold lines (such as dotted line 12 shown in FIG. 1) may be weakened compared to the surrounding material, for example by being thinner or comprising a different material, to facilitate folding. Flap 5 is then folded inwardly as indicated by arrow 13 onto the central portion of mat 3, following which flap 4 is folded inwardly onto the reverse side of flap 5, as indicated by arrow 14. At this point, the kit 1 is partially closed as shown in FIG. 3.

From the partially closed position, the lower end 15 of mat 3 is folded inwardly as indicated by arrow 16 onto the folded-over flaps 4, 5 (see FIG. 3). Finally, the folded-over upper end of mat 3 is then folded inwardly as indicated by arrow 17 and the two hook-and-loop fastener tabs 2a (reverse side of) and 2b brought together to close the kit 1 as shown in FIG. 1. The kit 1 now encloses a soiled nappy and used wipes.

Thus on completion of a nappy change the soiled nappy and used wipes may be enclosed in the kit 1 to form a waste bag which can be disposed of. Alternatively, the waste bag can be kept until a suitable disposal means is available. In some cases, it may be desirable to place the waste bag into a waterproof disposal bag optionally provided with the kit 1 to avoid leakage from a very soiled nappy.

In another embodiment, the pockets on flap 4 could be switched to flap 5 to make the kit easier for left-handed carers to use.

In a further embodiment, flap 4 or flap 5 comprises one or more further pockets with items such as baby cream and/or hand cream and/or a waterproof disposal bag.

In a different embodiment, a further layer of material (such as a pad) is provided over the nappy-changing surface of the mat 3. This pad would provide an initial surface on which a soiled nappy is taken off a baby. The pad could thereafter be removed from the rest of the mat 3 so that a cleaner mat surface is available for putting on a clean nappy. Alternatively, the pad could be removed prior to putting an outer garment onto the nappy (such as a “babygrow”).

The kit 1 may be produced in different sizes (for example small, medium and large) suitable for babies of different ages and sizes. When closed, as shown in FIG. 1, the kit 1 is preferably able to fit easily into a handbag or pocket or into a glove or side compartment of a vehicle.

The kit 1 is preferably produced from inexpensive, preferably biodegradable material. The kit 1 could be produced primarily from recycled material.

A second embodiment of the invention is shown in an open position shown in FIG. 4. The kit 21 is cruciform-shaped or (small) T-shaped (inverted) with an elongated mat 23 having extending therefrom side flaps (or mat extensions) 24, 25. Side flap 24 incorporates a first envelope or pocket 26 with one or more sheets of a material moistened with cleansing liquid (for example, “baby wipes”), and side flap 25 incorporates a second envelope or pocket 27 with baby cream or ointment. Each pocket 26, 27 can be opened with one hand using a tear-tab 28a, 28b located at one corner of each pocket 26, 27. An unused nappy 29 (shown in outline) fits loosely onto a central position of the mat 23 and is available when the kit 21 is open. The kit 21 also includes drying material 40 (shown in outline) and a separately sealed antiseptic wipe 41 (shown in outline) fitted loosely onto a central position of the mat 23, along with the unused nappy 29. At an upper end of the mat 23 is located a padded head rest 30 onto which a baby's head can be rested.

The kit 21 also has an optional detachable or removable layer of material 35 located at a lower end 36 of the mat 23. The layer of material 35 is preferably waterproof, as least one side.

In use, a basic nappy-changing kit 21 would first be opened as with first embodiment above. When open, the kit 21 provides a clean, hygienic, elongated working surface on mat 23. The unused nappy 29 is set aside from the mat 23 and the baby reclined onto mat 23 with his or her head resting on head rest 30. The two flaps 24, 25 provide a wider working surface area. The pockets 26, 27 are used to store materials used in changing the baby, and these pockets 26, 27 are easily opened with one hand using the tear-tabs 28a, 28b so that the carer has a free hand for attending to the baby, leaving the other hand to effect the cleaning and moving soiled items. A soiled nappy once removed from the baby can be conveniently placed to one side on flap 25. The baby can then be cleaned using baby wipes provided in pocket 26, and the used wipes placed to one side on the other flap 25. The baby cream provided in pocket 27 can be applied and the unused nappy can then be fitted onto the baby.

When finished with a nappy change, the baby is lifted and held against the body with one arm, leaving the other arm free to close the kit 21 by folding the mat 23 and flaps 24, 25 as described above for the first embodiment (with line 32 and arrows 31, 33 and 34 shown in FIG. 4 corresponding to line 12 and arrows 11, 13 and 14 shown in FIG. 2, respectively) and fastened using two hook-and-loop fastener tabs (reverse side of one tab 22a shown in FIG. 4).

In a variation of the above changing process, the optional layer of material 35 provides an initial surface onto which the baby with a soiled nappy is placed. The soiled nappy would be removed from the baby while the baby is positioned on the layer of material 35. The layer of material 35 could then be removed or detached from the mat 23 so that a cleaner surface provided by the mat 23 is available for putting on a clean nappy 29. Particularly if waterproof, the layer of material 35 could also provide a surface onto which soiled items can be placed away from the mat 23 until the nappy change is completed. The layer of material 35 could then be placed onto the mat 23 when the kit 21 is folded for disposal.

In a variation of the second embodiment shown in FIG. 4, flap 24 comprises one or more further pockets with items such as baby cream or ointment and antiseptic wipes for the carer to use after the changing process. Alternatively, the antiseptic wipe is provided in a separate sealed container to be used after the folding process is complete so that the carer can clean their hands before further attending to the baby.

The invention thus provides an all-inclusive single-use nappy-changing kit or packet for short-term, emergency or regular use. The kits are quick and easy to use, inexpensive to manufacture and purchase, conveniently dispensed from a vending machine, hygienic, lightweight, travel friendly and easily and readily disposable. A nappy change can take place almost anywhere as the packet forms its own clean surface without any fuss or special requirements and furthermore provides its own means for enclosing a soiled nappy and used nappy-changing material so that they can be safely discarded.

1. A single-use kit for hygienically changing and disposing of a baby's nappy, or diaper, comprising a foldable and/or rollable mat and a first mat extension having a pocket containing a nappy-changing material, in which the kit is openable by unfolding and/or unrolling the mat and first mat extension and closable by folding and/or rolling the mat and first mat extension, the kit when open providing (i) a nappy-changing surface on the mat and (ii) single-handed access to the nappy-changing material, and in which the kit when closed is suitable for enclosing a soiled nappy and/or used nappy-changing material.

2. The kit according to claim 1, comprising an unused nappy and/or further nappy changing material.

3. The kit according to claim 2, in which the unused nappy and/or further nappy changing material are positioned within the kit to be accessible when the kit is open.

4. The kit according to claim 1, in which the pocket is frangibly sealed to facilitate single-handed opening.

5. The kit according to claim 1, in which the pocket comprises a tear strip to facilitate single-handed opening.

6. The kit according to claim 1, in which the pocket comprises a tab to facilitate single-handed opening.

7. The kit according to claim 1, in which the mat is substantially rectangular when the kit is open.

8. The kit according to claim 1, in which the first mat extension extends from a side of the mat.

9. The kit according to claim 8, in which the kit comprises a second mat extension substantially opposite the first mat extension.

10. The kit according to claim 9, in which the kit when open is of cruciform shape.

11. The kit according to claim 9, in which the kit when open is T-shaped.

12. The kit according to claim 9, in which the kit is closed by folding and/or rolling the mat and mat extensions, and opened by unfolding and/or unrolling the mat and mat extensions, the second mat extension forming a surface for receiving a soiled nappy when the kit is open.

13. The kit according to claim 1, in which the nappy-changing material includes one or more sheets of liquid-absorbing material, and/or one or more sheets of material impregnated with a cleansing liquid, and/or one or more sheets of material impregnated with an antiseptic and/or antiseptic material, and/or a protective cream or lotion for a baby, and/or a hand cream, and/or a bag (for example, a sealable bag), and/or a baby toy.

14. The kit according to claim 1, in which the mat comprises a padded section onto which the baby's head can be placed when the kit is open.

15. The kit according to claim 1, comprising a fastener (for example, a releasable fastener such as a tie means and/or a hook-and-loop fastening means and/or reusable adhesive tabs) attached to the mat and/or either mat extension, in which the fastener allows the kit to be held closed.

16. The kit according to claim 1, comprising a carry means (for example, a handle).

17. The kit according to claim 1, in which the mat and/or mat extensions are formed from a plastics material.

18. The kit according to claim 1, in which the mat and/or mat extensions are formed from paper with a plastics coating.

19. The kit according to claim 1, in which the kit is substantially biodegradable.

20. The kit according to claim 1, in which the kit when closed has a waterproof outer surface.

21. The kit according to claim 1, in which the kit (for example, when closed) is suitable for vending from a vending machine.

22. The kit according to claim 21, in which the kit when closed is of a compact and/or robust design for vending from a vending machine.

23. The kit according to claim 1, in which the mat comprises a releasable layer (for example, a pad) covering at least part of the nappy-changing surface.

24. (canceled)

25. The kit according to claim 2, in which the further nappy-changing material includes one or more sheets of liquid-absorbing material, and/or one or more sheets of material impregnated with a cleansing liquid, and/or one or more sheets of material impregnated with an antiseptic and/or antiseptic material, and/or a protective cream or lotion for a baby, and/or a hand cream, and/or a bag (for example, a sealable bag), and/or a baby toy.
